(19) United States
(12) Patent Application Publication
(10) Pub. No.: US 2007/0015840 A1
(43) Pub. Date: Jan. 18, 2007

(54) Use of an alkyl ether of hydroxystilbene for the treatment of dry skin

(75) Inventors: Maria Dalko, Gif S/Yvette (FR); Gilles Rubinstenn, Paris (FR)

Correspondence Address: C. IRVIN MCCLELLAND;OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 10/538,662

(22) PCT Filed: Nov. 10, 2003
(86) PCT No.: PCT/EP03/12507

§ 371 Date: Aug. 7, 2006

Related U.S. Application Data
(60) Provisional application No. 60/438,775, filed on Jan. 9, 2003.

(30) Foreign Application Priority Data
Dec. 18, 2002 (FR) 0216113

(51) Int. Cl. A61K 31/075

(52) U.S. Cl. 514/720

(57) ABSTRACT

The present invention relates to a method for the cosmetic treatment of dry skin or of a dry scalp, comprising the topical application to the skin or the scalp of a composition containing, in a physiologically acceptable medium, at least one alkyl ether of hydroxystilbene with a saturated or unsaturated, linear or branched C1-C6 alcohol. The composition may be used for cosmetic purposes, for treating drying out of the skin, in particular after the menopause, or for dermatological purposes, for treating disorders associated with oligoseborrhoeic dry skin, in particular forms of dermatitis.

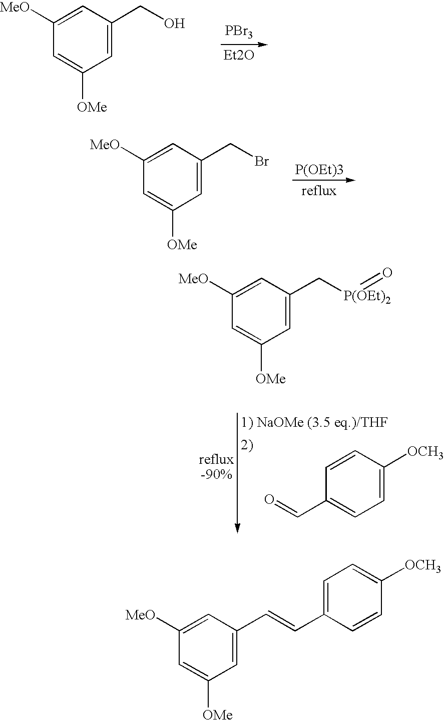

The present invention relates to a method for the cosmetic treatment of dry skin or of a dry scalp, comprising the topical application to the skin or the scalp of a composition containing, in a physiologically acceptable medium, at least one alkyl ether of hydroxystilbene with a saturated or unsaturated, linear or branched C₁-C₆ alcohol.

From the age of thirty-five, and more particularly after the menopause, many women frequently complain of having dry skin and of discomfort or unaesthetic effects resulting therefrom (desquamation, dull complexion, skin atony, facial tautness). This dryness is caused, as is now known, by a decrease in the production of sebum with age.

Moreover, children whose sebaceous function is not yet active often show signs of dry skin, which can progress to atopic dermatitis.

Sebum is the natural product of the sebaceous gland which, together with the sweat produced by the eccrine or apocrine glands, constitutes a natural moisturizer for the epidermis. It consists essentially of a more or less complex mixture of lipids. Conventionally, the sebaceous gland produces squalene, triglycerides, aliphatic waxes, cholesterol waxes and, possibly, free cholesterol (Stewart, M. E., Semin. Dermatol. 11, 100-105 (1992)). The action of bacterial lipases converts a variable portion of the triglycerides into free fatty acids.

The sebocyte is the competent cell of the sebaceous gland. The production of sebum is associated with the programme of terminal differentiation of this cell. During this differentiation, the metabolic activity of the sebocyte is essentially focussed on lipid biosynthesis (lipogenesis) and more precisely on the neosynthesis of fatty acids and of squalene.

A compound for stimulating the production of the lipids constituting sebum, by the cells of the sebaceous gland (the sebocytes), would therefore be of definite advantage in treating oligoseborrhoeic dry skin, i.e. skin exhibiting a sebum content of less than 100 μg/cm² on the forehead.

To this end, it was proposed, in U.S. Pat. No. 4,496,556, to use DHEA, a steroid secreted by the adrenal glands, or esters thereof, administered topically, to increase the production of sebum.

However, for regulatory reasons, it is not always possible to use this type of compound in the cosmetics field. In addition, it is not sufficiently effective on oligoseborrhoeic skin. There is thus still the need for cosmetically acceptable compounds for efficiently stimulating the sebaceous function with a view to treating oligoseborrhoeic dry skin.

The applicant has now discovered, surprisingly, that certain hydroxystilbene ethers make it possible to satisfy this need.

Hydroxystilbenes such as resveratrol and pinosylvin are stilbenes produced by plants, in particular grapevine (leaves, shoots, fruit) and plants of the Polygonum genus, in particular Polygonum cuspidatum. These compounds have in particular been described as being capable of reducing the adhesion of microorganisms to the skin and of being useful, as a result, in cosmetic or dermatological products intended to treat acne, dandruff or unpleasant odours, and more particularly in body hygiene products (EP-0 953 345). It has also been suggested to use them in combination with retinoids, for potentiating the effect of the latter, in particular with a view to lightening the skin (WO 01/43705).

Finally, document WO 03/055444 discloses a vast family of resveratrol analogues, comprising alkyl ethers, which can be used to treat signs of skin ageing, in particular by stimulating collagen synthesis and fibroblast proliferation.

However, to the applicant's knowledge, it has never yet been suggested that alkyl ethers of hydroxystilbenes could be useful in the treatment of dry skin, in particular of oligoseborrhoeic skin.

On the contrary, resveratrol has been described as an inhibitor of 5α-reductase and therefore naturally finds an application in the treatment of greasy skin (FR-2 816 843). In fact, the applicant verified that resveratrol decreased the ability of sebocytes to produce sebum.

Now, against all expectations, the applicant discovered that alkyl ethers of hydroxystilbenes increased the ability of sebocytes to produce sebum.

A subject of the present invention is therefore a method for the cosmetic treatment of dry skin or of a dry scalp, comprising the topical application to the skin or the scalp of a composition containing, in a physiologically acceptable medium, at least one alkyl ether of hydroxystilbene of formula (I): 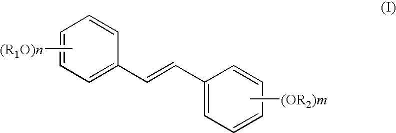
or its cis-isomer, in which R₁ and R₂ denote, independently, a saturated or unsaturated, linear or branched C₁-C₆ alkyl group, and
m and n are independently integers between 0 and 3, it being understood that m and n cannot simultaneously be zero.

A subject of the invention is also the cosmetic use of at least one alkyl ether of hydroxystilbene of formula (I), as defined above, as an agent for treating dry skin or a dry scalp.

The composition used according to the invention is particularly suitable for treating oligoseborrhoeic skin and an oligoseborrhoeic scalp, and it is therefore advantageously applied on individuals exhibiting a sebum content of less than 100 μg/cm², measured on the forehead, for example by means of the method described in FR-2 368 708.

The composition according to the invention makes it possible to restore the production of sebum by the sebocytes and, by the same token, to improve the comfort of dry skin and of a dry scalp.

It also makes it possible to combat the tautness of the skin and/or the dull and/or lifeless appearance of the skin and/or of the hair resulting from them drying out.

A subject of the invention is also the use of an alkyl ether of hydroxystilbene, as defined above, for preparing a composition, in particular a dermatological composition, intended to treat disorders associated with oligoseborrhoeic dry skin, in particular forms of dermatitis.

The compounds of formula (I) which are preferred for use in the present invention are those for which n=2 and m=0 or 1, i.e. the alkyl ethers of resveratrol and of pinosylvin, in particular the methyl ethers of these hydroxystilbenes, i.e. the compounds in which all the R₁ and R₂ groups denote a methyl radical.

The alkyl ethers of hydroxystilbenes according to the invention can be prepared according to synthetic processes consisting in using various coupling reactions, for example those known as Mc Murry (N. A. Ali, K. Kondo, Y. Tsuda, Chem. Pharm. Bull., 40(5), 1130-1136, (1992)), Wittig (N. A. Ali, K. Kondo, Y. Tsuda, Chem. Pharm. Bull., 40(5), 1130-1136, (1992)), Perkin (Spath E., Kromp K., Chem. Ber., 1941, 74, 189-192) and Heck (Synlett, 1998, 792) reactions.

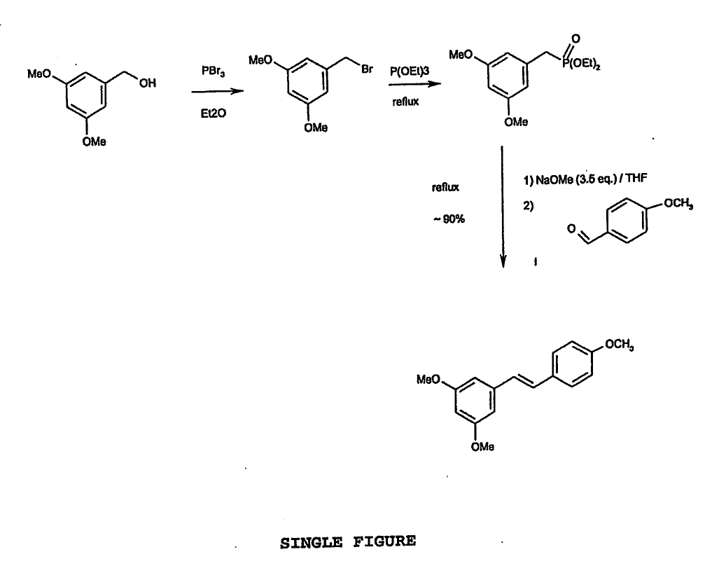

Resveratrol trimethyl ether can in particular be obtained by synthesis according to the process described in Phytochemistry, 24(7), 2309-12 (1998) and illustrated in FIG. 1.

According to this process, commercial dimethoxybenzyl alcohol is converted into the corresponding bromide, which is itself converted into diethyl phosphonate. The yield is 84% after purification and distillation. The key step in the synthesis is the Wittig-Horner reaction. The desired olefin is formed from the diethyl phosphonate and from para-methoxybenzaldehyde, in the presence of sodium methoxide in THF, with a yield of 88%, after purification by filtration on silica.

Pinosylvin dimethyl ether is, moreover, commercially available from the company APIN CHEMICALS.

The amount of alkyl ether of hydroxystilbene which can be used in the invention depends, of course, on the desired effect and may therefore vary within a large range. To give an order of magnitude, the alkyl ether of hydroxystilbene can be used in an amount representing from 0.001% to 5% of the total weight of the composition, preferably in an amount representing from 0.05% to 1% of the total weight of the composition.

The composition according to the invention is generally suitable for topical application to the skin and/or the scalp, and it therefore contains a physiologically acceptable medium, i.e. a medium which is compatible with the skin, its integuments (eyelashes, nails and hair) and/or the mucous membranes.

This composition may be in any presentation form normally used in cosmetics and dermatology, and it may in particular be in the form of an optionally gelled oily solution, a dispersion, optionally two-phase, of the lotion type, an emulsion obtained by dispersing a fatty phase in an aqueous phase (O/W) or inversely (W/O), or a triple emulsion (W/O/W or O/W/O) or a vesicular dispersion of the ionic and/or non-ionic type. These compositions are prepared according to the usual methods. A composition in the form of an oil-in-water emulsion is preferably used according to this invention.

This composition may be more or less fluid and may have the appearance of a white or coloured cream, an ointment, a milk a lotion, a serum, a paste or a mousse. It may optionally be applied in the form of an aerosol. It may also be in solid form, in particular in stick form. It may be used as a care product and/or a cleansing/makeup-removing and/or a makeup product for the skin. It may also be used as a shampoo or conditioner.

In a known manner, the composition used according to the invention may also contain adjuvants which are common in the cosmetics field, such as hydrophilic or lipophilic gelling agents, hydrophilic or lipophilic active agents, preserving agents, antioxidants, solvents, fragrances, fillers, screening agents, pigments, odour absorbers and colorants. The amounts of these various adjuvants are those conventionally used in the field under consideration, for example from 0.01 to 20% of the total weight of the composition. Depending on their nature, these adjuvants can be introduced into the fatty phase, into the aqueous phase or into the lipid vesicles. In any event, these adjuvants, and also the proportions thereof, will be chosen so as not to harm the desired properties of the alkyl ethers of hydroxystilbenes according to the invention.

When the composition used according to the invention is an emulsion, the proportion of the fatty phase may range from 5 to 80% by weight, and preferably from 5 to 50% by weight, relative to the total weight of the composition. The oils, emulsifiers and co-emulsifiers used in the composition in emulsion form are chosen from those conventionally used in the field under consideration. The emulsifier and the co-emulsifier are present in the composition in a proportion ranging from 0.3 to 30% by weight, and preferably from 0.5 to 20% by weight, relative to the total weight of the composition.

As oils which may be used in the invention, mention may be made of mineral oils (liquid petroleum jelly), oils of plant origin (avocado oil, soybean oil), oils of animal origin (lanolin), synthetic oils (perhydrosqualene), silicone oils (cyclomethicone) and fluoro oils (perfluoropolyethers). Fatty alcohols (cetyl alcohol), fatty acids and waxes (carnauba wax, ozokerite) may also be used as fatty substances.

As emulsifiers and co-emulsifiers which can be used in the invention, mention may, for example, be made of fatty acid esters of polyethylene glycol, such as PEG-100 stearate, and fatty acid esters of glycerol, such as glyceryl stearate.

Hydrophilic gelling agents which may be mentioned in particular include carboxyvinyl polymers (carbomer), acrylic copolymers such as acrylate/alkyl-acrylate copolymers, polyacrylamides, polysaccharides, natural gums and clays, and lipophilic gelling agents which may be mentioned include modified clays, such as bentones, metal salts of fatty acids, hydrophobic silica and polyethylenes.

As active agents, it will be advantageous to introduce into the composition used according to the invention at least one compound chosen from: desquamating agents; antibacterial agents; moisturizers; calmatives; and agents for stimulating keratinocyte proliferation and/or differentiation.

In fact, the stimulation of seborrhoea with the alkyl ethers of hydroxystilbenes according to the invention may, in certain individuals, provide a terrain of proliferation for the resident microflora of the follicular ostium (in particular Propionibacterium acnes), thus giving rise to considerable hydrolysis of the triglycerides of the sebum into free fatty acids and the reduction of the unsaturations of the polyunsaturated fatty acids (in particular linoleic acid). These two phenomena may contribute towards keratinization of the infundibulum and to the formation of a microcomedone. This may degenerate into a comedone, producing unaesthetic blockage and dilation of the pore. At a more advanced stage, this blockage may change into an inflammatory acneic lesion.

The addition of desquamating agents or agents regulating keratinocyte proliferation or differentiation to the composition according to the invention makes it possible to avoid the formation of these comedones. Similarly, antibacterial or bacteriostatic agents would make it possible to obtain the same effect, by modifying the proliferation of the resident microflora.

In addition, the moisturizers may supplement the effect obtained using the alkyl ethers of hydroxystilbenes according to the invention, and the calmatives are useful for improving the level of comfort of oligoseborrhoeic dry skin.

Examples of such additional active agents are given below.

Desquamating Agents

The term “desquamating agent” is intended to mean any compound capable of acting:

either directly on the desquamation by promoting exfoliation, such as β-hydroxy acids, in particular salicylic acid and its derivatives (including 5-n octanoyl salicylic acid); α-hydroxy acids, such as glycolic acid, citric acid, lactic acid, tartaric acid, malic acid or mandelic acid; urea; gentisic acid; oligofucoses; cinnamic acid; extract of Saphora japonica; resveratrol;

or on the enzymes involved in the desquamation or degradation of the corneodesmosomes, glycosidases, stratum corneum chymotryptic enzyme (SCCE), or even other proteases (trypsin, chymotrypsin-like). Mention may be made of agents for chelating mineral salts: EDTA; N-acyl-N,N′,N′-ethylenediaminetriacetic acid; aminosulphonic compounds and in particular (N-2-hydroxyethylpiperazine-N-2-ethane)sulphonic acid (HEPES); derivatives of 2-oxothiazolidine-4-carboxylic acid (procysteine); derivatives of alpha-amino acids of the glycine type (as described in EP-0 852 949), and sodium methylglycinediacetate marketed by BASF under the trade name Trilon M); honey, sugar derivatives such as O-octanoyl-6-D-maltose and N-acetylglucosamine.

Moisturizer

The term “moisturizer” is intended to mean:

either a compound acting on the barrier function, in order to maintain the moisturization of the stratum corneum, or an occlusive compound. Mention may be made of ceramides, sphingoid-based compounds, lecithins, glycosphingolipids, phospholipids, cholesterol and its derivatives, phytosterols (stigmasterol, β-sitosterol or campesterol), essential fatty acids, 1,2-diacylglycerol, 4-chromanone, pentacyclic triterpenes such as ursolic acid, petroleum jelly and lanolin;

or a compound which directly increases the water content of the stratum corneum, such as threalose and its derivatives, hyaluronic acid and its derivatives, glycerol, pentanediol, sodium pidolate, serine, xylitol, sodium lactate, polyglyceryl acrylate, ectoin and its derivatives, chitosan, oligosaccharides and polysaccharides, cyclic carbonates, N-lauroylpyrrolidonecarboxylic acid and N-α-benzoyl-L-arginine;

or a compound which activates the sebaceous glands, such as DHEA and its derivatives and vitamin D and its derivatives.

Agents for Stimulating Keratinocyte Proliferation and/or Differentiation

The agents for stimulating keratinocyte proliferation which can be used in the composition according to the invention comprise in particular phloroglucinol; the walnut cake extracts marketed by the company Gattefosse; and the Solanum tuberosum extracts marketed by the company Sederma.

The agents for stimulating keratinocyte differentiation comprise, for example, minerals such as calcium; the extract of lupin marketed by the company Silab under the trade name Photopreventine®; sodium beta-sitosteryl sulphate marketed by the company Seporga under the trade name Phytocohesine®; and the extract of corn marketed by the company Solabia under the trade name Phytovityl®.

Calmatives

Among the raw materials which are effective as calmatives, mention may be made, in a non-limiting manner, of the following active agents: pentacyclic triterpenes, such as β-glycyrrhetinic acid, its salts and/or its derivatives (glycyrrhetinic acid monoglucuronide, stearyl glycyrrhetinate, 3-stearoyloxyglycyrrhetic acid), ursolic acid and its salts, oleanolic acid and its salts, betulinic acid and its salts; extracts of Paeonia suffruticosa and/or lactiflora, of Rosmarinus officinalis, of epilobium, of Pygeum, of Boswellia serrata, of Centipeda cunnighami, of Helianthus annuus, of Cola nitida, of clove and of Bacopa moniera; salicylic acid salts and in particular zinc salicylate; extracts of algae, in particular of Laminaria saccharina; Canola oil, omega-3-unsaturated oils such as musk rose oil, blackcurrant oil, ecchium oil, fish oil; α-bisabolol and extracts of camomile; allantoin; the phosphoric diester of vitamin E and C; capryloylglycine; tocotrienols; piperonal; aloe vera; phytosterols; strontium salts; spring waters and in particular the spring water of the Vichy basin and the spring water of La Roche Posay; bacterial extracts and in particular the extract of non-photosynthetic filamentous bacteria described in patent application EP-0 761 204, preferably prepared from bacteria belonging to the order Beggiatoales, and more particularly a strain of Vitreoscilla filiformis; an extract of cells (preferably undifferentiated cells) of at least one plant from the Iridacea family, obtained by in vitro culturing, preferably an aqueous extract of Iris pallida, as described in particular in patent application EP-0 765 668; an extract of a plant of the Rosaceae family, preferably cultivated in vivo, advantageously of the species Rosa gallica, more preferably a water-glycol extract of Rosa gallica petals, as described in particular in patent application EP-0 909 556.

Antibacterial Agents

The antibacterial agents which can be used in the present invention may in particular be chosen from 2,4,4′-trichloro-2′-hydroxydiphenyl ether (or triclosan), 3,4,4′-trichlorobanilide, phenoxyethanol, phenoxypropanol, phenoxyisopropanol, undecylenic acid and its salts, 3-hydroxybenzoic acid, 4-hydroxybenzoic acid, phytic acid, N-acetyl-L-cystein acid, lipoic acid, azelaic acid and its salts, arachidonic acid, 2,4,4′-trichloro-2′-hydroxydiphenyl ether, 3,4,4′-trichlorocarbanalide, octopirox, octoxyglycerine, octanoylglycine, caprylyl glycol, 10-hydroxy-2-decanoic acid, dichlorophenyl imidazol dioxolane and its derivatives, described in patent WO 93/18743, farnesol and phytosphingosines, and mixtures thereof.

Preferably, the composition used according to the invention does not comprise any retinoid.

The invention will now be illustrated with the following non-limiting examples. In these examples, the amounts are indicated as percentages by weight.

### EXAMPLES

### Example 1

### Demonstration of the Activity of the Alkyl Ethers of Hydroxystilbenes on Lipogenesis

Resveratrol trimethyl ether was tested on a model of immortalized human sebocytes in culture, derived from the SZ95 line described in Zouboulis, C. C., Seltmann, H., Neitzel, H. & Orfanos, C. E., Establishment and Characterization of an Immortalized Human Sebaceous Gland Cell Line, J. Invest. Dermatol., 113, 1011-1020 (1999).

The test consisted in measuring the amount of lipids produced by the sebocytes of the line (at confluence), in the presence or absence of an active agent diluted in DMSO, at two different concentrations, such that the final amount of DMSO in the culture medium is 0.1% and the amount of resveratrol trimethyl ether is 0.01% (4×10⁻⁴ M) and 0.001% (4×10⁻⁵ M), respectively. After treatment for 24 hours, the adherent cells are treated with Nile Red (1 μg/ml). The lipid content is then quantified by measuring the fluorescence of the dye (two excitation/emission pairs: 485-540 nm for the neutral lipids and 540-620 nm for the non-neutral lipids). The results are given for the total lipids (combination of the two measurements).

The experiment is performed in sextuplicate (products assayed and control) in 96-well plates and repeated four times.

The results are given in the table below:

| Concentration of | VARIATION IN | |
|---|---|---|
| resveratrol | LIPIDS (relative | P |
| trimethyl ether | to the control) | (Student's test) |
| 0.01% | +46% | 0.004 |
| 0.001% | +36% | 0.009 |

As emerges from this table, the resveratrol trimethyl ether induces a significant increase in the sebocytic lipogenesis. In comparison, resveratrol, tested under the same conditions and at the same concentrations, significantly inhibits the lipogenesis, respectively by 20% and 67%.

### Example 2

### Cosmetic Composition

This composition is prepared in a manner that is conventional for those skilled in the art. The amounts given in these examples are indicated as percentages by weight.

| | |
|---|---|
| Resveratrol trimethyl ether | 0.5% |
| 5-n-octanoylsalicylic acid | 1% |
| Methylparaben | 0.1% |
| Propylparaben | 0.1% |
| Lanolin | 5% |
| Liquid petroleum jelly | 4% |
| Sesame oil | 4% |
| Cetyl alcohol | 5% |
| Glyceryl monostearate | 2% |
| Triethanolamine | 1% |
| Propylene glycol | 5% |
| Carbomer 940 | 0.1% |
| Water | qs 100% |

This cream, applied twice daily, makes it possible to revive the radiance of dry skin.

1-20. (canceled)

21. A method for cosmetically treating dry skin or dry scalp, comprising:
topically applying a composition to one or both of the skin or the scalp of a human,
wherein the composition comprises:
a physiological acceptable medium, and
at least one hydroxystilbene alkyl ether of formula (I) or a cis-isomer thereof: 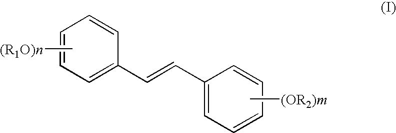
wherein R₁ and R₂ are each independently a saturated or unsaturated, linear or branched C₁-C₆ alkyl group, and
m and n are each 0, 1, 2 or 3, wherein m and n are not both 0.

22. The method according to claim 21, wherein n=2 and m=0 or 1.

23. The method according to claim 21, wherein each of R₁ and R₂ are methyl groups.

24. The method according to claim 21, wherein the hydroxystilbene alkyl ether is present in the composition in amount of from 0.05% to 1% by weight based on the total weight of the composition.

25. The method according to claim 21, wherein the composition does not contain any retinoid.

26. The method according to claim 21, wherein the composition further comprises at least one desquamating agent.

27. The method according to claim 26, wherein the desquamating agent is at least one selected from the group consisting of salicylic acid, a derivative of salicylic acid, an α-hydroxy acid, urea, gentisic acid, an oligofucose, cinnamic acid, an extract of Saphora japonica, resveratrol, EDTA, N-acyl-N,N′,N′-ethylenediaminetriacetic acid, an aminosulphonic compound, a derivative of 2-oxothiazolidine-4-carboxylic acid, a derivative of a glycine α-amino acid, honey and a sugar derivative.

28. The method according to claim 26, wherein the desquamating agent is at least one select from the group consisting of 5-n-octanoyl salicylic acid, glycolic acid, citric acid, lactic acid, tartaric acid, malic acid, mandelic acid, (N-2-hydroxyethylpiperazine-N-2-ethane) sulfonic acid, sodium methylglycinediacetate, O-octanoyl-6-D-maltose, and N-acetylglucosamine

29. The method according to claim 21, wherein the composition further comprises a moisturizer.

30. The method according to claim 29, wherein the moisturizer is at least one selected from the group consisting of a ceramide, a sphingoid compound, a lecithin, a glycosphingolipid, a phospholipid, a cholesterol, a derivative of a cholesterol, a phytosterol, an essential fatty acid, 1,2-diacylglycerol, 4-chromanone, pentacyclic triterpene, a threalose, a derivative of a threalose, hyaluronic acid, a derivative of hyaluronic acid, glycerol, pentanediol, sodium pidolate, serine, xylitol, sodium lactate, polyglyceryl acrylate, ectoin, a derivative of ectoin, chitosan, an oligosaccharide, a polysaccharide, a cyclic carbonate, N-lauroylpyrrolidonecarboxylic acid, N-α-benzoyl-L-arginine, DHEA, a derivative of DHEA, vitamin D, and a derivative of vitamin D.

31. The method according to claim 29, wherein the moisturizer at least one selected from the group consisting of stigmasterol, beta-sitosterol, campesterol, ursolic acid, petroleum jelly and lanolin.

32. The method according to claim 21, wherein the composition further comprises at least one calmative.

33. The method according to claim 32, wherein the calmative is at least one select from the group consisting of a pentacyclic triterpene, ursolic acid, a salt of ursolic acid, oleanolic acid, a salt of oleanolic acid, betulinic acid, a salt of betulinic acid, an extract of Paeonia suffruticosa, and extract of lactiflora, an extract of Rosmarinus officinalis, an extract of epilobium, an extract of Pygeum, an extract of Boswellia serrata, an extract of Centipeda cunnighami, an extract of Helianthus annuus, an extract of Cola nitida, an extract of clove, an extract of Bacopa moniera, a salicylic acid salt, an extract of an algae, Canola oil, omega-3-unsaturated oil, an alpha-bisabolol, an extract of chamomile, allantoin, a phosphoric diester of vitamin E, a phosphoric diester of vitamin C, capryloylglycine, a tocotrienol, piperonal, aloe vera, a phytosterol, a strain of Vitreoscilla filiformis, an aqueous extract of Iris pallida, and a water-glycol extract of Rosa gallica petals.

34. The method according to claim 32, wherein the calmative is at least one selected from the group consisting of beta-glycyrrhetinic acid, a salt of beta-glycyrrhetinic acid, a derivative of beta-glycyrrhetinic acid, zinc salicylate, an extract of Laminaria saccharina, musk rose oil, blackcurrant oil, ecchium oil, and fish oil.

35. The method according to claim 21, wherein the composition further comprises at least one antibacterial agent.

36. The method according to claim 35, wherein the antibacterial agent is at least one selected from the group consisting of triclosan, phenoxyethanol, octoxyglycerine, octanoylglycine, 10-hydroxy-2-decanoic acid, caprylyl glycol, farnesol and azelaic acid.

37. The method according to claim 21, wherein the composition further comprises at least one agent for stimulating keratinocyte proliferation, stimulating keratinocyte differentiation or stimulating both keratinocyte proliferation and keratinocyte differentiation.

38. The method according to claim 21, wherein the composition is in the form of an oil-in-water emulsion.

39. The method according to claim 21, wherein the composition is applied to a human having a sebum content of less than 100 μg/cm² on the forehead.

40. The method according to claim 21, wherein the composition is applied in an amount effective for at least one of alleviating the tautness of skin, alleviating a dull appearance of skin, alleviating a lifelessness appearance of the skin or alleviating a lifelessness appearance of the hair.

41. The method according to claim 21, wherein the composition is applied in an amount effective for treating the dry skin or the dry scalp.

42. The method of claim 21, wherein the composition is applied in an amount effective for increasing sebocytic lipogenesis.

43. The method of claim 21, wherein the composition comprises resveratrol trimethylether and the applying induces an increase in sebocytic lipogenesis.

44. The method of claim 21, wherein the composition comprises resveratrol trimethylether and the composition is applied in an amount effective for inducing an increase in sebocytic lipogenesis.

45. The method of claim 21, wherein the composition is applied to the skin or the scalp for treating one or more disorders associated with oligoseborrhoeic dry skin.

46. The method according to claim 45, wherein the disorder is a form of a dermatitis.
